(19) United States
(12) Patent Application Publication
(10) Pub. No.: US 2025/0380871 A1
(43) Pub. Date: Dec. 18, 2025

(54) NON-CONTACT CLINICAL RAMAN SPECTROSCOPY GUIDED PROBE AND APPLICATIONS OF SAME

(71) Applicants: VANDERBILT UNIVERSITY, Nashville, TN (US); THE BOARD OF TRUSTEES OF THE UNIVERSITY OF ILLINOIS, Urbana, IL (US)

(72) Inventors: Sean Fitzgerald, Nashville, TN (US); Anita Mahadevan-Jansen, Nashville, TN (US); Stephen Boppart, Urbana, IL (US); Guillermo Monroy, Urbana, IL (US); Jay A. Werkhaven, Nashville, TN (US)

(21) Appl. No.: 18/877,391

(22) PCT Filed: Jun. 20, 2023
(86) PCT No.: PCT/US2023/025777

§ 371 Date: Dec. 20, 2024

Related U.S. Application Data
(63) Continuation-in-part of application No. 17/691,567, filed on Mar. 10, 2022.
(60) Provisional application No. 63/158,962, filed on Mar. 10, 2021.
(60) Provisional application No. 63/353,737, filed on Jun. 20, 2022.

(51) Int. Cl. A61B 5/00

(52) U.S. Cl. CPC A61B 5/0075; A61B 5/0037; A61B 5/0084

(57) ABSTRACT

A non-contact clinical Raman spectroscopy guided probe includes at least one first fiber operably coupled with a first light source and having a working end for delivering excitation light emitted from the first light source to the target site; at least one second fiber operably coupled with a detector and having a working end for collecting Raman scattering light scattered from the target site in response to excitation by the excitation light to the detector; and a lens positioned between the working ends of the at least one first fiber and the at least one second fiber and the target site for focusing the excitation light onto the target site and retrieving collection efficiency of the Raman scattering light. The probe is also guided during device positioning to provide the user with feedback on orientation in the lateral and axial directions to repeatably measure specific locations on the target site.

### CROSS-REFERENCE TO RELATED PATENT APPLICATIONS

This PCT application claims priority to and the benefit of U.S. Provisional Patent Application Ser. No. 63/353,737, filed Jun. 20, 2022, which is incorporated herein by reference in its entirety.

This PCT application is also a continuation application of U.S. patent application Ser. No. 17/691,567, filed Mar. 10, 2022, which claims priority to and the benefit of U.S. Provisional Patent Application Ser. No. 63/158,962, filed Mar. 10, 2021, which are incorporated herein by reference in their entireties.

### STATEMENT AS TO RIGHTS UNDER FEDERALLY-SPONSORED RESEARCH

This invention was made with government support under Grant No. R01EB028615 awarded by the National Institutes of Health. The government has certain rights in the invention.

### FIELD OF THE INVENTION

The invention relates generally to Raman spectroscopy in biomedical applications, and more particularly, to a non-contact clinical Raman spectroscopy guided probe and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

The emergence of label-free optical spectroscopy for biomedical analysis has offered a clinically compatible set of methods to probe molecular changes in tissue related to disease. The interaction of light and its prescribed properties (e.g., wavelength, polarization, and phase) with primary absorbers and fluorophores allows for a quantitative measure of their changing concentration and conformation. Methods like diffuse reflectance spectroscopy, autofluorescence, and vibrational spectroscopy (i.e., infrared and Raman) exemplify the diversity of spectroscopic tools available to non-invasively probe tissue composition. A large body of research has demonstrated that Raman spectroscopy (RS) provides higher specificity than any other form of label-free optical spectroscopy for disease classification. Unlike absorption- or fluorescence-based spectroscopy methods which are most sensitive to a small subset of the primary chromophores and fluorophores, respectively, RS provides detection of discrete molecular signals from all tissue components, in both ex vivo and in vivo settings.

RS utilizes the inelastic scattering interaction to analyze the presence and abundance of biomolecules relating to sample composition. Inelastic scattering is a weak optical interaction, so collecting spectra through spontaneous production of Raman scattered light requires extremely sensitive detection hardware and relatively high excitation laser power and acquisition times compared to other optical sensing methods. Raman scattered light is collected with either free space optics or fiber optic probes and is then spectrally resolved with high efficiency spectrometers.

While forward-firing RS probes are useful in that they can be designed with small probe diameters to analyze tissues on or within body cavities, they optimally analyze Raman spectra when the fiber optics directly contact the sample. However, when the fiber optic is removed from contacting the sample, diffuse emissions are less able to couple back into the fiber for detecting the weak Raman scattered light. Also, the light exiting the laser fiber begins diverging and covers a larger area of the tissue during non-contact acquisition, exciting multiple spatial regions and therefore losing spatial specificity of the Raman signal.

Therefore, a heretofore unaddressed need exists in the art to address the aforementioned deficiencies and inadequacies.

### SUMMARY OF THE INVENTION

In view of the foregoing, one of the objectives of this invention is to provide a novel in-vivo Raman spectroscopy (RS) probe for use in analyzing tissues within small body cavities, which cannot be directly contacted, for spectral acquisition.

In one aspect, the invention relates to a probe for collecting Raman signal of a target site of interest, which can be oral cavity and lymphoid tissues, middle ear tissues, cervical, gastrointestinal, esophageal and nasal tissues, or the like. The probe includes at least one first fiber operably coupled with a first light source and having a working end for delivering excitation light emitted from the first light source to the target site; at least one second fiber operably coupled with a detector and having a working end for collecting Raman scattering light scattered from the target site in response to excitation by the excitation light to the detector; and a lens positioned between the working ends of the at least one first fiber and the at least one second fiber and the target site for focusing the excitation light onto the target site and retrieving collection efficiency of the Raman scattering light.

In one embodiment, the at least one second fiber includes a plurality of second fibers spatially arranged surrounding the at least one first fiber.

In one embodiment, the at least one first fiber and the plurality of second fibers are spatially arranged in a row, a matrix, a wing, or a ring form.

In one embodiment, the plurality of second fibers spatially is arranged in a radial ring form originated from the at least one first fiber.

In one embodiment, the lens is adapted to minimize optical signal contribution from the lens itself that does not interfere with the Raman signature from the target site while maintaining a small outer diameter.

In one embodiment, the lens is positioned at a distance of 2F from the working end of the at least second fiber, thereby providing an imaging relay with a working distance of 2F from the probe tip, wherein F is a focal length of the lens.

In one embodiment, the lens includes a quartz lens, a sapphire lens, a calcium fluoride (CaF₂) lens, or a lens formed of any material whose inherent signal does not interfere with the Raman signals of the target site.

In one embodiment, the probe further comprises a first optical filter placed at the working end of the at least first fiber and a second optical filter placed at the working end of the at least second fiber, respectively.

In one embodiment, the first optical filter is a short-pass or band-pass optical filter that blocks all wavelengths longer than that of the excitation light; and the second optical filter is a long-pass or band-notch optical filter that blocks all wavelengths equal to or shorter than that of the excitation light, thereby preventing backscattered excitation light from being collected by the at least second fiber.

In one embodiment, the first optical filter and the second optical filter are configured such that the probe is capable of measuring dual regions of the Raman scattering light from the target site, wherein the dual regions include a fingerprint (FP) region and a high-wavenumber (HW) region.

In one embodiment, the dual regions of the Raman scattering light are sequentially acquired by switching the excitation light between a first wavelength and a second wavelength, wherein the first wavelength and the second wavelength are adapted such that when excited by the first wavelength light, the Raman scattering light corresponds to the FP region; and when excited by the second wavelength light, the Raman scattering light corresponds to the HW region. In one embodiment, the first wavelength is in a range of about 630-1064 nm, and the second wavelength is in a range of about 570-900 nm.

In one embodiment, the first wavelength is about 785 nm, and the second wavelength about 680 nm, and wherein the short pass optical filter has a cut-off wavelength at about 785 nm, and the long pass filter has cut-on wavelength at about 800 nm.

In one embodiment, the first wavelength is about 830 nm, and the second wavelength about 710 nm, and wherein the short pass optical filter has a cut-off wavelength at about 830 nm, and the long pass filter has cut-on wavelength at about 850 nm.

In one embodiment, the first optical filter and the second optical filter are configured such that the probe is capable of measuring the Raman scattering light in a high-wavenumber (HW) region from the target site.

In one embodiment, the lens includes a glass lens.

In one embodiment, the probe further comprises a guidance mechanism for performing range sensing and providing feedback on orientation and position of the probe in lateral and axial directions to repeatably measure specific locations on the target site.

In one embodiment, the guidance mechanism comprises a low-coherence interferometry (LCI) detector having at least one third fiber operably coupled with a second light source and having a working end for delivering low coherence light emitted from the second light source to the target site, wherein the at least one third fiber is located next to the at least one first fiber such that both beams of the excitation light and the low coherence light share the lens and are co-localized on the target site, ensuring that the LCI feedback is co-registered to the position of the excitation light spot.

In one embodiment, the guidance mechanism comprises a miniature camera module located at the probe tip for providing wide-field visualization of the lateral position of the probe relative to the target site, wherein the miniature camera module includes a camera and an illumination fiber that delivers broadband light to visualize an imaging field of the camera.

In one embodiment, the probe of is configured to measure the Raman scattering light without contact to the target site.

In another aspect, the invention relates to a system for assessment of a target site of interest, which can be oral cavity and lymphoid tissues, middle ear tissues, cervical, gastrointestinal, esophageal and nasal tissues, or the like. The system comprises a first light source configured to operably emit excitation light; and a probe comprising at least one first fiber operably coupled with the first light source and having a working end for delivering the excitation light emitted from the first light source to the target site; at least one second fiber having a working end for collecting Raman scattering light scattered from the target site in response to excitation by the excitation light; and a lens positioned between the working ends of the at least one first fiber and the at least one second fiber and the target site for focusing the excitation light onto the target site and retrieving collection efficiency of the Raman scattering light.

In one embodiment, the system further comprises a detector coupled with the probe for obtaining a plurality of Raman spectra from the collected Raman scattering light, wherein each Raman spectrum is associated with biomolecular content of a spot of the target site at which the Raman scattering light is scattered, and wherein the plurality of Raman spectra is processed to identify spectral features and assess the target site from the identified spectral features.

In one embodiment, the system further comprises a controller operably coupled with the detector and configured to process the plurality of Raman spectra so as to identify spectral features and assess the target site from the identified spectral features.

In one embodiment, the system further comprises a display operably coupled with the controller for displaying the plurality of Raman spectra, the identified spectral features, and/or the assessment of the target site.

In one embodiment, the first light source comprises a single wavelength laser module configured to operably emit the excitation light of a single wavelength, or a dual wavelength laser module configured to be operably emit the excitation light of a wavelength switchable between a first wavelength and a second wavelength.

In one embodiment, the at least one second fiber includes a plurality of second fibers spatially arranged surrounding the at least one first fiber.

In one embodiment, the at least one first fiber and the plurality of second fibers are spatially arranged in a row, a matrix, a wing, or a ring form.

In one embodiment, the plurality of second fibers spatially is arranged in a radial ring form originated from the at least one first fiber.

In one embodiment, the lens is adapted to minimize optical signal contribution from the lens itself that does not to interfere with Raman signature from the target site while maintaining a small outer diameter.

In one embodiment, the lens is positioned at a distance of 2F from the working end of the at least second fiber, thereby providing an imaging relay with a working distance of 2F from the probe tip, wherein F is a focal length of the lens.

In one embodiment, the lens includes a quartz lens, a sapphire lens, a calcium fluoride (CaF₂) lens, or a lens formed of any material whose inherent signal does not interfere with the Raman signals of the target site.

In one embodiment, the probe further comprises a first optical filter placed at the working end of the at least first fiber and a second optical filter placed at the working end of the at least second fiber, respectively.

In one embodiment, the first optical filter is a short-pass or band-pass optical filter that blocks all wavelengths longer than that of the excitation light; and the second optical filter is a long-pass or band-notch optical filter that blocks all wavelengths equal to or shorter than that of the excitation light, thereby preventing backscattered excitation light from being collected by the at least second fiber.

In one embodiment, the first optical filter and the second optical filter are configured such that the probe is capable of measuring dual regions of the Raman scattering light from the target site, wherein the dual regions include a fingerprint (FP) region and a high-wavenumber (HW) region.

In one embodiment, the dual regions of the Raman scattering light are sequentially acquired by switching the excitation light between the first wavelength and the second wavelength, wherein the first wavelength and the second wavelength are adapted such that when excited by the first wavelength light, the Raman scattering light corresponds to the FP region; and when excited by the second wavelength light, the Raman scattering light corresponds to the HW region.

In one embodiment, the first wavelength is in a range of about 630-1064 nm, and the second wavelength is in a range of about 570-900 nm.

In one embodiment, the first wavelength is about 785 nm, and the second wavelength about 680 nm, and wherein the short pass optical filter has a cut-off wavelength at about 785 nm, and the long pass filter has cut-on wavelength at about 800 nm.

In one embodiment, the first wavelength is about 830 nm, and the second wavelength about 710 nm, and wherein the short pass optical filter has a cut-off wavelength at about 830 nm, and the long pass filter has cut-on wavelength at about 850 nm.

In one embodiment, the first optical filter and the second optical filter are configured such that the probe is capable of measuring the Raman scattering light in a high-wavenumber (HW) region from the target site.

In one embodiment, the lens includes a glass lens.

In one embodiment, the probe further comprises a guidance mechanism for performing range sensing and providing feedback on orientation and position of the probe in lateral and axial directions to repeatably measure specific locations on the target site.

In one embodiment, the guidance mechanism comprises a low-coherence interferometry (LCI) detector having at least one third fiber operably coupled with a second light source and having a working end for delivering low coherence light emitted from the second light source to the target site, wherein the at least one third fiber is located next to the at least one first fiber such that both beams of the excitation light and the low coherence light share the lens and are co-localized on the target site, ensuring that the LCI feedback is co-registered to the position of the excitation light spot.

In one embodiment, the guidance mechanism comprises a miniature camera module located at the probe tip for providing wide-field visualization of the lateral position of the probe relative to the target site, wherein the miniature camera module includes a camera and an illumination fiber that delivers broadband light to visualize an imaging field of the camera.

In one embodiment, the probe of is configured to measure the Raman scattering light without contact to the target site.

In yet another aspect, the invention relates to non-transitory tangible computer-readable medium storing instructions which, when executed by one or more processors, cause the system to deliver excitation light emitted from the first light source to the target site; collect Raman scattering light scattered from the target site in response to excitation by the excitation light; obtain a plurality of Raman spectra from the collected Raman scattering light; and process the plurality of Raman spectra so as to identify spectral features and assess the target site from the identified spectral features.

In one embodiment, the instructions, when executed by the one or more processors, further cause the system to display the plurality of Raman spectra, the identified spectral features, and/or the assessment of the target site.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only. The drawings are not intended to limit the scope of the present teachings in any way.

FIG. 1A shows Raman signal collection by a standard probe in contact and non-contact mode (15 mm offset) acquisition, and an improved probe utilizing a stock lens to collect the spectrum during the non-contact mode.

FIG. 1B shows collection efficiencies from a chemical standard (i.e., polypropylene) between the standard probe in contact and non-contact mode (15 mm offset) acquisition, and the improvement when utilizing a stock lens to collect the spectrum during the non-contact mode as shown in FIG. 1A.

FIG. 2 shows optical signal contributions specifically in the FP region of Raman signals from different lenses when excited with light at a wavelength of about 785 nm. These would change depending on the light wavelength.

FIG. 3A shows schematically a non-contact clinical Raman spectroscopy guided probe according to embodiments of the invention.

FIG. 3B shows schematically a cross sectional view at the working ends of the excitation fiber and collection fibers of the non-contact clinical Raman spectroscopy guided probe shown in FIG. 3A.

FIG. 4 shows schematically a non-contact clinical Raman spectroscopy guided probe according to embodiments of the invention.

FIG. 5 shows schematically a system having non-contact clinical Raman spectroscopy guided probe according to embodiments of the invention.

FIG. 6A shows schematically a standard RS probe and a lensed RS-LCI probe positioned 12 mm above an optical phantom composed of bone overlaying a milk solution to mimic a composite biological sample with spatially-specific Raman signals. As depicted, the probes were laterally translated in 0.5 mm increments for acquiring Raman spectra. As used herein, the term ‘optical phantom’ refers to a sample that is designed to mimic some aspect of true tissues. In this example, a composite sample was created with bone and milk (which have distinct Raman signals) to test the spatial selectivity of the RS-LCI lensed probe.

FIG. 6B shows a ratio of the bone-specific peak at 960 cm⁻¹ to a milk-specific peak at 1130 cm⁻¹ against the offset of the standard RS probe and the lensed RS-LCI probe from the bone.

FIG. 6C shows Raman spectra acquired by the standard probe at each lateral position, color-coded from red to blue.

FIG. 6D shows Raman spectra acquired by the RS-LCI probe at each lateral position, color-coded from red to blue according to embodiments of the invention.

FIG. 7 shows the FP (response to the first wavelength) and HW (response to the second wavelength) spectra of various oral tissues from a human subject acquired by the RS-LCI probe according to embodiments of the invention. In this embodiment, Raman spectra were generated with 785 nm excitation for the FP and 680 nm for the HW.

FIG. 8 shows the FP and HW spectra from two example cases, one with ear fluid and one without ear fluid acquired by the RS-LCI probe according to embodiments of the invention. In this embodiment, Raman spectra were generated with 785 nm excitation for the FP and 680 nm for the HW.

FIG. 9 shows schematically signal collection improvement for the FP and HW spectra acquired by the lensed probe according to embodiments of the invention over a standard probe. The spectra were collected by measuring a fingernail of a human subject with 785 nm excitation for the FP spectrum and 680 nm for the HW spectrum. Spectra were measured at the same working distance from the sample (i.e., 12 mm) and with identical acquisition settings (i.e., 2 sec exposure time, 3 accumulations) and laser power (i.e., 50 mW power measured out of probe tip).

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that, as used in the description herein and throughout the claims that follow, the meaning of “a”, “an”, and “the” includes plural reference unless the context clearly dictates otherwise. Also, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, “charge-coupled device” or “CCD” refers to an analog shift register that enables the transportation of analog signals (electric charges) through successive stages (capacitors), controlled by a clock signal. Charge-coupled devices can be used as a form of memory or for delaying samples of analog signals. Today, they are most widely used in arrays of photoelectric light sensors to serialize parallel analog signals. In a CCD for capturing images, there is a photoactive region (an epitaxial layer of silicon), and a transmission region made out of a shift register (the CCD, properly speaking).

An image is projected through a lens onto the capacitor array (the photoactive region), causing each capacitor to accumulate an electric charge proportional to the light intensity at that location. A one-dimensional array, used in line-scan cameras, captures a single slice of the image, while a two-dimensional array, used in video and still cameras, captures a two-dimensional picture corresponding to the scene projected onto the focal plane of the sensor. Once the array has been exposed to the image, a control circuit causes each capacitor to transfer its contents to its neighbor (operating as a shift register). The last capacitor in the array dumps its charge into a charge amplifier, which converts the charge into a voltage. By repeating this process, the controlling circuit converts the entire semiconductor contents of the array to a sequence of voltages, which it samples, digitizes and stores in some form of memory.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

One of the objectives of this invention is to provide non-contact Raman spectroscopy (RS) probe that circumvents many of the limitations of standard forward-firing fiber optic probes used for RS, specifically when the handheld probe operates in non-contact mode. While forward-firing RS probes are useful in that they can be designed with small probe diameters (e.g., 1-10 mm) to analyze tissues on or within body cavities, they optimally analyze Raman spectra when the fiber optics directly contact the sample.

When a fiber optic is removed from contacting the sample, diffuse emissions are less able to couple back into the fiber for detecting the weak Raman scattered light. Also, the diverging light exiting the laser fiber begins diverging and covers a larger area of the tissue, exciting multiple spatial regions and therefore loses spatial specificity of the Raman signal. The limitations imposed by non-contact applications of RS probes are summarized as:

(1) Reduction in collection efficiency when the collection fibers are not directly contacting the sample. This is especially impactful for RS probes, as Raman scattering is a weak optical effect that requires relatively high laser powers and long acquisition times compared to other optical techniques.

(2) Deteriorating spatial specificity of RS data. This is due to the diverging nature of light exiting the laser fiber. When the device is offset from the sample, a larger area of the sample is illuminated with laser light, and therefore back-scattered Raman emissions will be averaged over the entire excited zone.

In one aspect of the invention, the non-contact RS probe is designed to collect RS optical signals when the probe does not contact the target tissue, which is particularly useful in small body cavities, which cannot be directly contacted, for spectral acquisition. The probe in some embodiments includes a custom miniature lens to reimage the Raman fiber bundle onto the surface of the target tissue at a determined working distance, for example, but is not limited to, a chosen distance of 12 mm. In one embodiment, the lens is, but not limited to, a single crystalline lens. The single crystalline lens that does not interfere with the detected Raman signature from the sample while maintaining a small outer diameter, e.g., of about 1-10 mm. This allows for retrieval of the collection efficiency that is lost when the probe is not put in direct contact with the target tissue, as well as focusing the excitation source to a spot for spatially-isolated Raman signal.

The non-contact RS probe is also guided during device positioning to provide the user with feedback on orientation in the lateral and axial directions to repeatably measure specific locations on the sample. Probe guidance is given by either a miniature camera, optical interferometric imaging, or both.

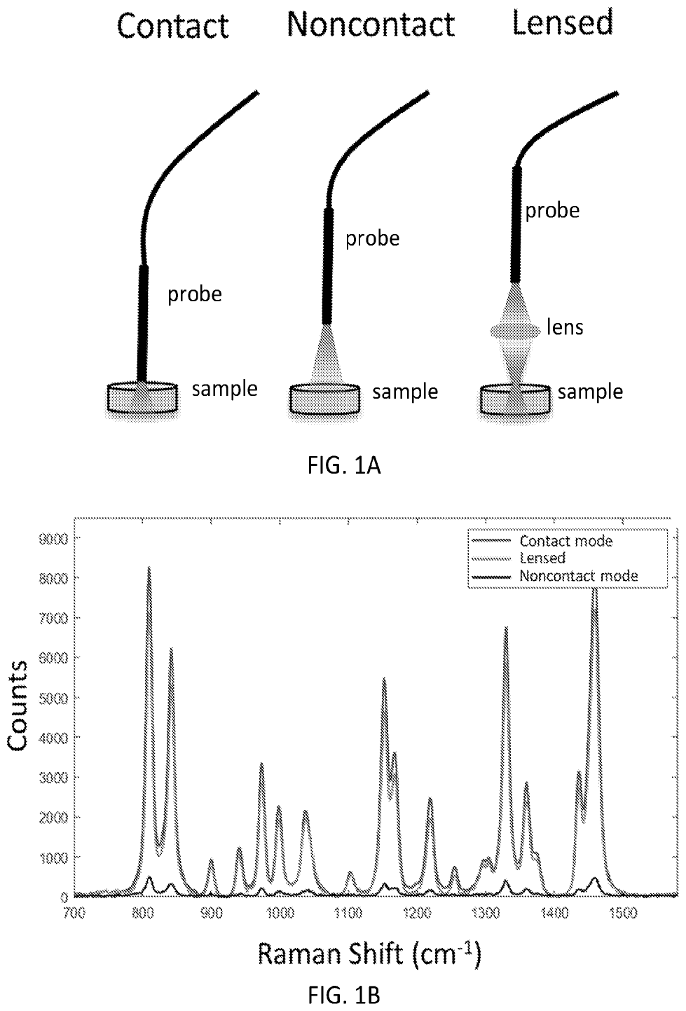

The motivation for using a lensed probe design to address limitation (1) and improve collection efficiency are summarized in the pictorial and experimental evidence in FIGS. 1A-1B, outlining the difference in collection efficiency from a chemical standard (i.e., polypropylene) between a standard probe in contact and non-contact mode (15 mm offset) acquisition, and the improvement when utilizing a stock lens to collect the spectrum during non-contact mode.

These results demonstrate that a lens helps to recover the lost collection efficiency related to non-contact acquisition and bring the spectral energy back to what is seen in contact-mode.

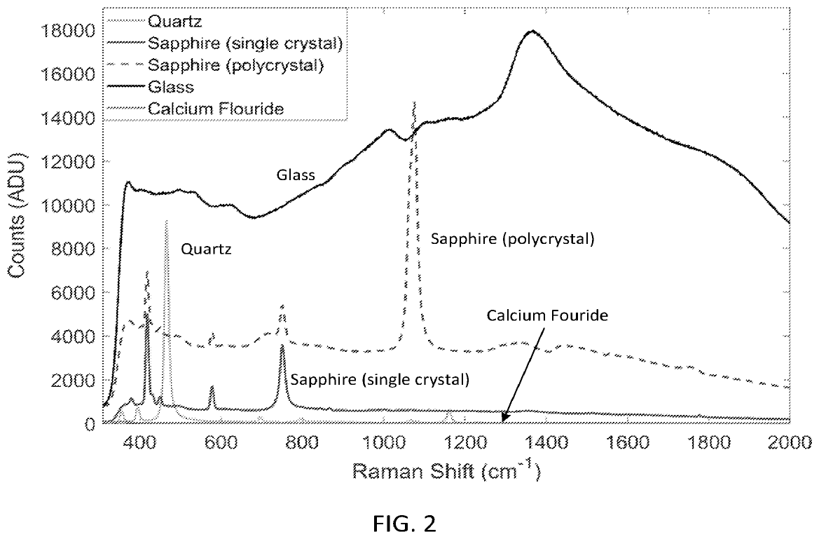

However, biological materials provide vastly less Raman signal than synthetic polymers, and a standard glass lens generates a strong interfering signal (FIG. 2, black trace) that becomes dominant in samples with weak Raman signal. So, a specialized RS probe has been designed to address this, which utilizes a crystalline lens that minimizes the optical signal contribution from the lens itself, demonstrated in the following experimental comparison of RS data from various lens materials:

In this comparison, as listed Table 1, glass lens (FIG. 2, black trace), quartz lens (FIG. 2, green trace), single-crystal (monocrystalline) sapphire lens (FIG. 2, solid blue trace), poly-crystal sapphire lens (FIG. 2, dotted blue trace) and calcium fluoride lens (FIG. 2, red trace) performed optimally in that these crystals contribute the least FP signal in the spectral range between 400-2000 cm⁻¹. It is shown that the grade of crystal has a strong effect on the optical response of the lens material.

TABLE 1
Comparison of optical signal contributions from different lenses

| Material | Refractive Index | Transmission | Raman peaks (cm⁻¹) |
|---|---|---|---|
| Glass | 1.52 | 91.7% | Broad Luminescence |
| Sapphire | 1.76 | 85.4% | 410, 640, 750 |
| Quartz | 1.54 | 91.2% | 700, 800, 1060, 1160 |
| Calcium | 1.43 | 93.8% | 321 |
| Fluoride | | | |

Another aspect of the invention is that the non-contact RS probe is designed to measure two distinct regions of the Raman spectrum, named the fingerprint (FP, 400-1800 cm⁻¹) and the high-wavenumber (HW, 2500-3800 cm⁻¹) regions. These provide complementary information on sample biochemical composition and can be sequentially acquired by switching the excitation laser line so that the associated spectral range detected by the spectrometer is shifted to acquire each region. For example, the FP region is detected with a spectrometer designed to measure between 800-950 nm when a 785 nm laser excites the sample. With the same spectrometer, the HW range is detected when excited with a 680 nm laser. It should be noted that any arbitrary set of laser lines can be utilized to practice this invention, so the probe include any variant of laser lines used for dual-region RS operation.

Forward-firing fiber probes require optical filters at the tips of the excitation and collection fibers, and dual-region detection requires a specific choice of optical filters:

Laser fiber filter is a short-pass or band-pass optical filter that blocks all wavelengths longer than the excitation beam. The purpose is to block extraneous emissions generated by the fiber itself from reaching the sample.

Collection fiber filter is a long-pass or band-notch optical filter to block the strong laser light from being collected and entering the spectrometer.

Optical coherence tomography (OCT), is an interferometric optical technique that is well-known for its ability to image sample microstructure. Often thought of as an optical analog to ultrasound, it provides cross-sectional depth scans of sample morphology on the micron-scale. While image features alone provide a wealth of useful information, integrating RS adds the ability to analyze chemical content within the sample. Relevant to probe guidance, OCT imaging can serve to help position the RS probe relative to the tissue in situations where probe positioning is a challenge, like within small body cavities. The potential for integrating these two techniques has been reported by many groups and recently summarized in a review article by the inventors, but no miniature designs exist that could be applied to screening in-vivo tissues within small body cavities for clinical diagnostics or similar applications that are spatially restricted.

The inventors have also recently reported an attempt to combine RS excitation/collection fibers and OCT imaging into a handheld device designed specifically for application in the middle ear. However, the previously mentioned limitations in collecting Raman spectra with fiber optics in non-contact mode limited system performance and spawned the device concept described in this patent submission.

Toward the development of a miniature RS-OCT probe for applications in clinical diagnostics, some embodiments of the device described in this patent utilize non-scanning OCT, termed low-coherence interferometry (LCI). This does not provide 2-dimensional images of tissue structure that are available in traditional OCT, but still provides range-sensing capabilities and gives the user with feedback on the distance between probe tip and sample surface when positioning the device. Signal detection and processing between OCT and LCI are identical, and the only difference is if the beam is optically scanned. The benefit of this approach in a non-contact RS probe is to control probe offset for reliably and repeatably acquiring Raman spectra.

These and other aspects of the invention are further described below. Many modifications and variations are possible in light of the teaching of the disclosure without departing from the spirit and scope of the novel concepts of the invention.

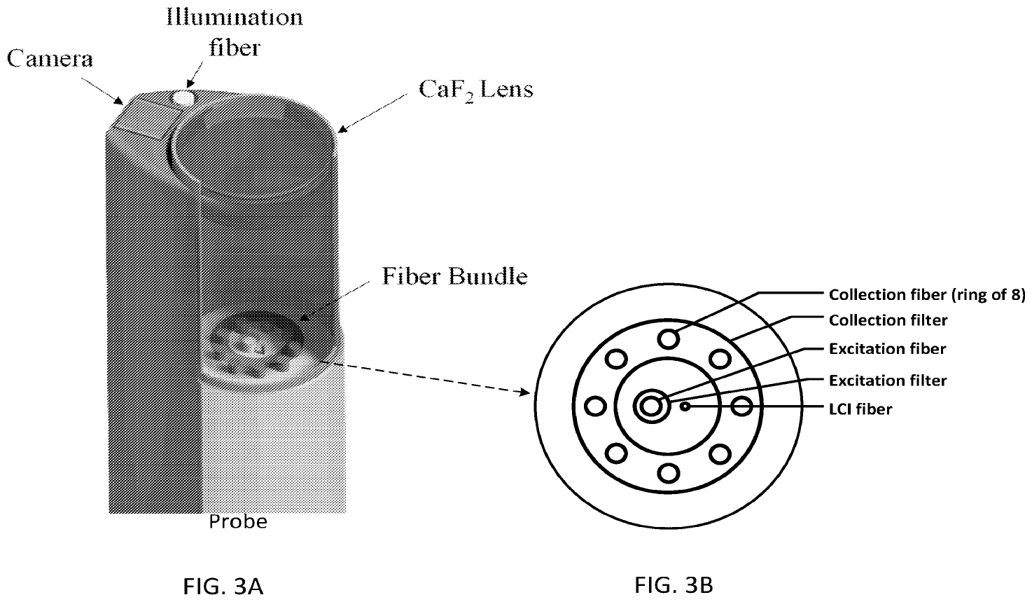

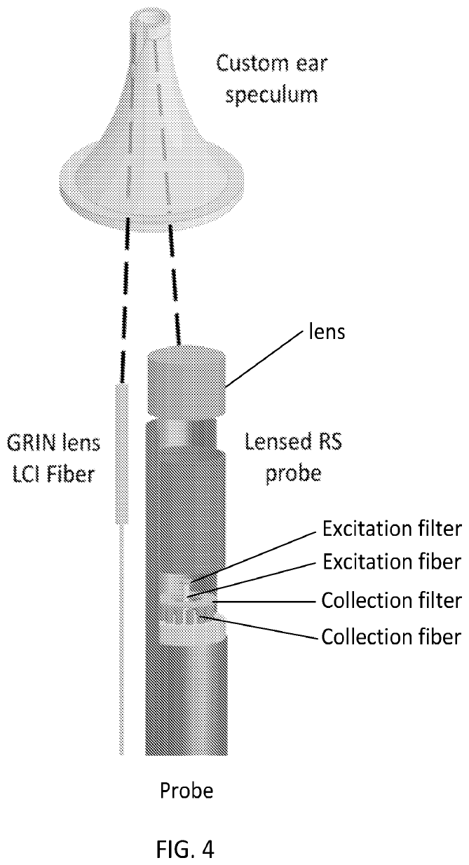

In some embodiments, as shown in FIGS. 3A-3B and 4, the non-contact RS probe for collecting Raman signal of a target site of interest, which can be oral cavity and lymphoid tissues, middle ear tissues, cervical, gastrointestinal, esophageal and nasal tissues, or the like, includes at least one first fiber (i.e., excitation fiber) operably coupled with a first light source and having a working end for delivering excitation light emitted from the first light source to the target site; at least one second fiber (i.e., collection fiber) operably coupled with a detector and having a working end for collecting Raman scattering light scattered from the target site in response to excitation by the excitation light to the detector; and a crystalline lens (e.g., CaF₂ lens) positioned between the working ends of the at least one first fiber and the at least one second fiber and the target site for focusing the excitation light onto the target site and retrieving collection efficiency of the Raman scattering light. It should be appreciated that a lens formed of any material whose inherent signal does not interfere with biological Raman signals of the target site of interest can be utilized to practice the invention. In addition, it should be noted that the term “first light source”, used herein, refers to, but is not limited to, a coherent light source, e.g., a laser source, capable of emitting coherent light with a single wavelength or coherent light with a narrowband (e.g., less than 1 nm bandwidth), while the term “second light source” refers to a wideband or broadband light source capable of emitting light with low coherence (e.g., greater than 100 nm bandwidth) or no coherence. Furthermore, it should be noted that the term “retrieving collection efficiency” refers to “recover collection efficiency” by using a lens. The lens allows the probe to provide the same collection efficiency as if the probe was in contact with the sample. This is demonstrated by the experimental data in FIGS. 3A-3B. The second benefit of the lens is confining the excitation light to a focus, thereby giving spatial selectivity in what part of the sample is excited during non-contact acquisition.

In some embodiments, the at least one second fiber includes a plurality of second fibers spatially arranged surrounding the at least one first fiber.

In some embodiments, the at least one first fiber and the plurality of second fibers are spatially arranged in a row, a matrix, a wing, or a ring form as shown in FIGS. 3A-3B and 4.

In some embodiments, the plurality of second fibers spatially is arranged in a radial ring form originated from the at least one first fiber, as shown in FIGS. 3A-3B and 4.

In some embodiments, the lens is adapted to minimize optical signal contribution from the lens itself that does not to interfere with Raman signature from the target site while maintaining a small outer diameter.

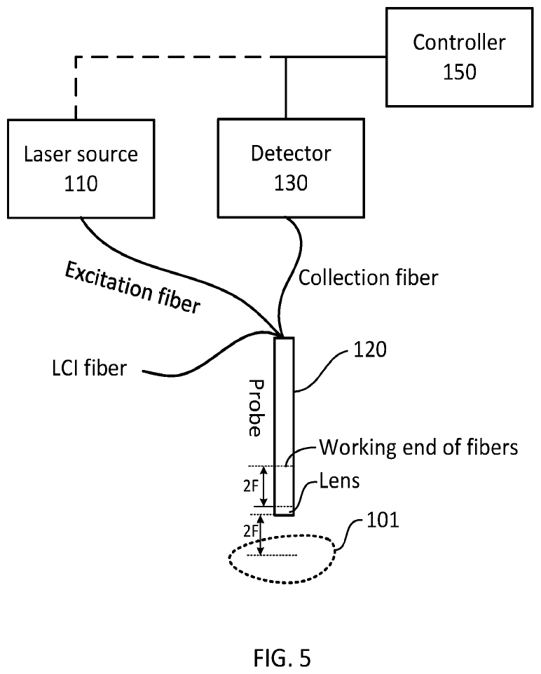

In some embodiments, as shown in FIG. 5, the lens is positioned at a distance of 2F from the working end of the at least second fiber, thereby providing an imaging relay with a working distance of 2F from the probe tip, wherein F is a focal length of the lens.

In some embodiments, the lens includes a quartz lens, a sapphire lens, a calcium fluoride (CaF₂) lens, or a lens formed of any material whose inherent signal does not interfere with the Raman signals of the target site.

In some embodiments, the probe further comprises a first optical filter (i.e., excitation filter shown in FIGS. 3A-3B and 4) placed at the working end of the at least first fiber and a second optical filter (i.e., collection filter shown in FIGS. 3A-3B and 4) placed at the working end of the at least second fiber, respectively.

In some embodiments, the first optical filter is a short-pass or band-pass optical filter that blocks all wavelengths longer than that of the excitation light; and the second optical filter is a long-pass or band-notch optical filter that blocks all wavelengths equal to or shorter than that of the excitation light, thereby preventing backscattered excitation light from being collected by the at least second fiber.

In some embodiments, the first optical filter and the second optical filter are configured such that the probe is capable of measuring dual regions of the Raman scattering light from the target site, wherein the dual regions include a fingerprint (FP) region and a high-wavenumber (HW) region.

In some embodiments, the dual regions of the Raman scattering light are sequentially acquired by switching the excitation light between a first wavelength and a second wavelength, wherein the first wavelength and the second wavelength are adapted such that when excited by the first wavelength light, the Raman scattering light corresponds to the FP region; and when excited by the second wavelength light, the Raman scattering light corresponds to the HW region.

In some embodiments, the first wavelength is in a range of about 630-1064 nm, and the second wavelength is in a range of about 570-900 nm.

In some embodiments, the first wavelength is about 785 nm, and the second wavelength about 680 nm, and wherein the short pass optical filter has a cut-off wavelength at about 785 nm, and the long pass filter has cut-on wavelength at about 800 nm.

In some embodiments, the first wavelength is about 830 nm, and the second wavelength about 710 nm, and wherein the short pass optical filter has a cut-off wavelength at about 830 nm, and the long pass filter has cut-on wavelength at about 850 nm.

In one embodiment, the first optical filter and the second optical filter are configured such that the probe is capable of measuring the Raman scattering light in a high-wavenumber (HW) region from the target site.

In one embodiment, the lens includes a glass lens. An amorphous glass lens interferes with detecting the FP region, but less of an issue for HW region. In one embodiment, the probe that only measures HW Raman spectra using a glass lens.

In some embodiments, the probe further comprises a guidance mechanism for performing range sensing and providing feedback on orientation and position of the probe in lateral and axial directions to repeatably measure specific locations on the target site.

In some embodiments, the guidance mechanism comprises a low-coherence interferometry (LCI) detector having at least one third fiber operably coupled with a second light source and having a working end for delivering low coherence light emitted from the second light source to the target site, wherein the at least one third fiber is located next to the at least one first fiber such that both beams of the excitation light and the low coherence light share the lens and are co-localized on the target site, ensuring that the LCI feedback is co-registered to the position of the excitation light spot.

In some embodiments, the guidance mechanism includes a dedicated optical fiber and gradient index (GRIN) lens to perform interferometric sensing, termed low coherence interferometry (LCI), as shown in FIG. 4. In this way, the device provide the user with a depth-resolved reflectance profile of any material in front of the device. This LCI contrast is used to provide feedback on the distance between probe tip and target tissue. The benefit of this approach is to control probe offset for reliably and repeatably acquire spectra in non-contact mode.

In some embodiments, the guidance mechanism comprises a miniature camera module located at the probe tip for providing wide-field visualization of the lateral position of the probe relative to the target site, wherein the miniature camera module includes a camera and an illumination fiber that delivers broadband light to visualize an imaging field of the camera.

In some embodiments, the probe of is configured to measure the Raman scattering light without contact to the target site.

In another aspect, the invention relates to a system, as shown in FIG. 5, for assessment of a target site of interest, which can be oral cavity and lymphoid tissues, middle ear tissues, cervical, gastrointestinal, esophageal and nasal tissues, or the like. The system comprises a first light source (i.e., laser source) 110 configured to operably emit excitation light; and a non-contact RS probe 120 as disclosed above for collecting Raman scattering light scattered from the target site 101 in response to excitation by the excitation light.

The probe 120 in some embodiments is schematically shown in FIGS. 3A-3B and 4 and has at least one excitation fiber operably coupled with the laser source 110 and having a working end for delivering excitation light emitted from the laser source 110 to the target site 101; at least one collection fiber having a working end for collecting Raman scattering light scattered from the target site 101 in response to excitation by the excitation light; and at least one LCI fiber operably coupled with a second light source and having a working end for delivering low coherence light emitted from the second light source to the target site 101. Referring to FIGS. 3A-3B and 4, the probe 120 also includes a lens positioned between the working ends of the at least one excitation fiber and the at least one collection fiber and the target site 101 for focusing the excitation light onto the target site and retrieving collection efficiency of the Raman scattering light.

In some embodiments, the system further comprises a detector 130 coupled with the probe 120 for obtaining a plurality of Raman spectra from the collected Raman scattering light, wherein each Raman spectrum is associated with biomolecular content of a spot of the target site 101 at which the Raman scattering light is scattered, and wherein the plurality of Raman spectra is processed to identify spectral features and assess the target site from the identified spectral features. The detector in certain embodiments can be one or more CCD cameras, one or more complementary metal oxide semiconductor (CMOS) cameras, one or more photosensor arrays, or a spectrograph.

In some embodiments, the system further comprises a controller 150 configured to process the plurality of Raman spectra so as to identify spectral features and assess the target site from the identified spectral features. The controller 150 can be a computer, or a device having one or more processors, or a microcontroller unit (MCU) for processing the plurality of Raman spectra.

In some embodiments, the system further comprises a display operably coupled with the controller for displaying the plurality of Raman spectra, the identified spectral features, and/or the assessment of the target site.

In some embodiments, the spectral features are associated with water, or electrolytes, or metabolic products, pus, or bacteria, or cells, and include spectral peaks in the high wavenumber region and the fingerprint region.

In some embodiments, the first light source comprises a single wavelength laser module configured to operably emit the excitation light of a single wavelength, or a dual wavelength laser module configured to be operably emit the excitation light of a wavelength switchable between a first wavelength and a second wavelength.

In yet another aspect, the invention relates to non-transitory tangible computer-readable medium storing instructions which, when executed by one or more processors, cause the system to deliver excitation light emitted from the first light source to the target site; collect Raman scattering light scattered from the target site in response to excitation by the excitation light; obtain a plurality of Raman spectra from the collected Raman scattering light; and process the plurality of Raman spectra so as to identify spectral features and assess the target site from the identified spectral features.

In one embodiment, the instructions, when executed by the one or more processors, further cause the system to display the plurality of Raman spectra, the identified spectral features, and/or the assessment of the target site.

The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

In one exemplary example, a custom Python application synchronizes data acquisition from the RS spectrometer with software-driven triggering of the light sources. This provides a level of automation for the acquisition of FP/HW Raman spectra, and LCI scans, by eliminating the need for the user to manually control light sources. The illumination fiber and LCI source fiber, used to visualize RS-LCI probe placement, cannot be active during the acquisition of Raman spectra due to the impact of these sources on the acquired RS data.

A second aspect of this software provides the user with real-time processing of the optical spectra to isolate and visualize the RS component of the signal. This allows for quality checking of the data while it is being acquired. The user interface allows for defining acquisition settings, processing parameters, and control of the light sources. Pre-defined triggering schemes can then be programed into dedicated buttons to provide synchronized control of the RS-LCI probe during acquisition in-vivo.

So application-driven switching between light sources (1) simplifies experimental protocol, (2) reduces user errors during spectral acquisition and (3) expedites measurements.

Without intent to limit the scope of the invention, exemplary embodiments according to the embodiments of the invention are given below.

One embodiment of the device has been fabricated and is described herein and depicted in FIGS. 3A-3B. A miniature lens composed of Calcium Fluoride (CaF₂) was designed with a 6 mm focal length (F) and 4 mm outer diameter. As shown in FIG. 5, when positioned at a distance of 2F from the fiber bundle, this provides an imaging relay with a working distance of 12 mm from the device tip. The fiber bundle in this inception of the non-contact RS probe was designed to operate at 785/680 nm for dual-region detection. It includes one multimode optical fiber with 0.22 numerical aperture (NA) to deliver the excitation laser light, affixed with a 785 nm short-pass laser filter overtop the fiber optic (FIG. 3A, green). This is surrounded by 8 collection fibers with 0.22 NA, affixed with an annular-shaped 800 nm long-pass collection filter (FIG. 3A, red).

For the probe guidance, a dedicated LCI fiber (FIG. 3A, blue) is located next to the RS laser fiber to perform range sensing and control the axial position of the device. In this way, both the RS and LCI beams share a common lens and are co-localized on the sample, ensuring that LCI feedback is co-registered to the position of the RS laser spot. A miniature camera module is also added to the design, which provides wide-field visualization of the lateral position of the probe relative to the sample. The module holds a commercially available endoscopic camera (1 mm diameter) and a dedicated illumination fiber that delivers broadband light to visualize the camera's imaging field.

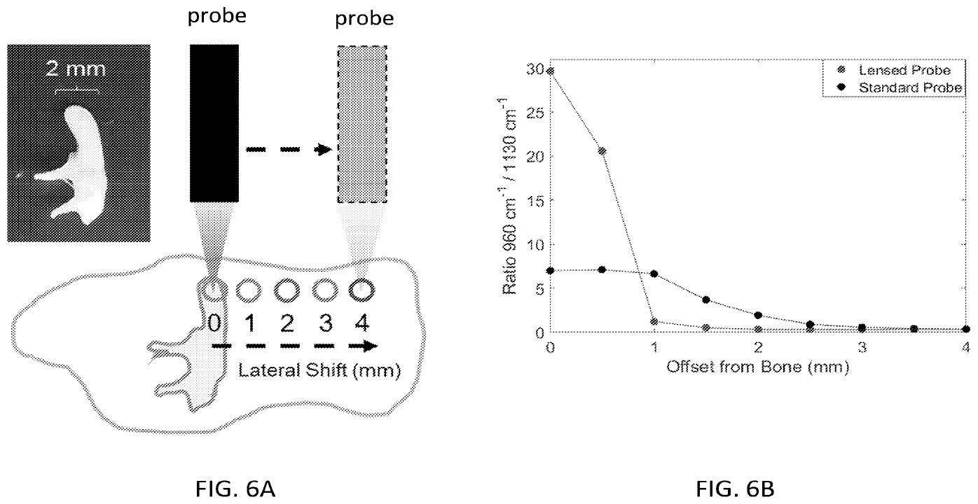

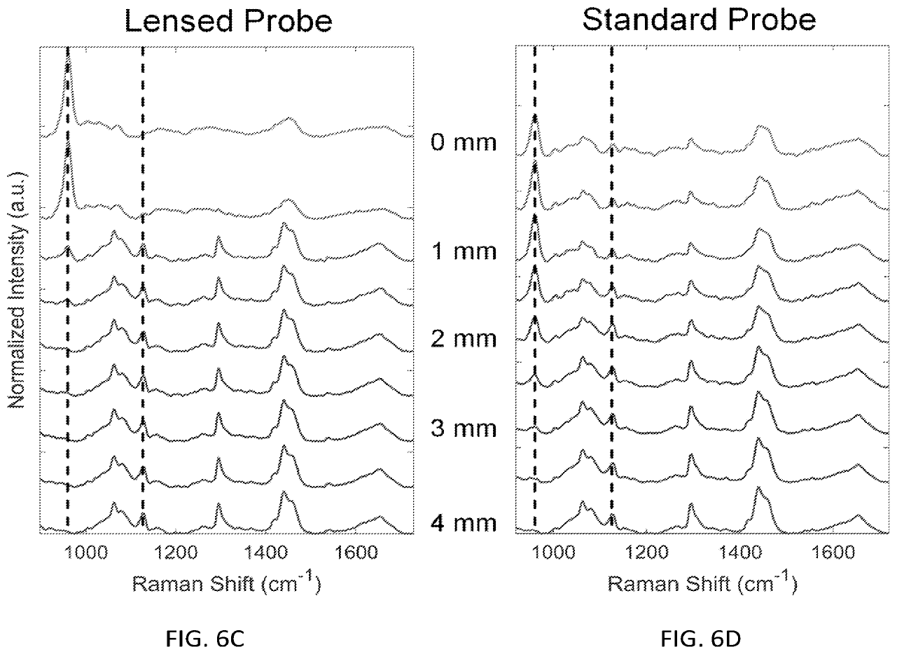

Towards limitation (2) of a non-contact RS probe, a simple biological model was generated to demonstrate that the focused RS laser spot using a miniature lens improves the ability to select specific Raman signals in the imaging field. Depicted in FIG. 6A, a bone sample was suspended over a solution of milk. Each of these biological materials have unique Raman signatures and served as a benchmark for spatial selectivity. A standard RS probe and the lensed RS-LCI probe were positioned 12 mm above the bone, and laterally translated in 0.5 mm increments. FIG. 6B shows schematically a ratio of the bone peak at 960 cm⁻¹ to a bone peak at 1130 cm⁻¹ against the offset of the standard RS probe and the lensed RS-LCI probe from the bone. The Raman spectra acquired by the standard probe and the RS-LCI probe at each lateral position, color-coded from red to blue, are shown in FIGS. 6C and 6D, respectively. These experimental results show that the prominent bone peak at 960 cm⁻¹ is immediately eliminated as the focused beam from the RS-LCI probe is translated off of the bone at the 1 mm position. The standard probe, however, continuously shows bone-related Raman features due to the diverging laser light covering a large area of the biological model.

The above exemplary embodiments are presented only for the purposes of illustration and description of the invention. Different variations are also apparent in light of the above teaching. For example, although small outer probe diameter (1-10 mm) to allow the probe the measure tissues or samples within small body cavities, the probe is not limited to only tissue analysis and can be used for analysis of other materials.

Miniature lens relay at the tip of the probe is used to focus the Raman laser onto the sample and retrieve collection efficiency for non-contact RS analysis. Any ‘Raman-silent’ lens material can be used for the miniature lens, including quartz, sapphire, or calcium fluoride. Any variant in focal length of the lens design can be used for the probe.

Probe guidance by either optical interferometry (OCT/LCI), widefield camera, or both.

Any variation in the second light source used for OCT/LCI, and the narrowband RS excitation laser wavelength.

Any variation of fiber tip filters can be used to perform dual-region RS analysis of FP and HW regions. For example, variation (1): 785/680 nm excitation laser: excitation filter—short pass optical filter at 785 nm, and collection filter—long pass filter at 800 nm; and variation (2): 830/710 nm excitation laser: excitation filter—short pass optical filter at 830 nm, and collection filter—long pass filter at 850 nm.

Any variation in the arrangement or number of excitation or collection fibers used to irradiate the sample and collect Raman emissions can also be utilized to practice the invention.

Potential applications for the invention would include any situation where the RS probe cannot directly contact the target sample and/or the user is occluded from viewing the position of the probe relative to sample. Some examples include, but are not limited to, oral cavity and lymphoid tissues (adenoids, tonsils, back of tongue, etc.), middle ear tissues (ear drum, ossicles, middle ear effusions, etc.), endocervical canal (vaginal wall, cervix, uterus, etc.), esophageal and nasal tissues (nasal cavity, nasopharynx, esophagus, etc.), or the like.

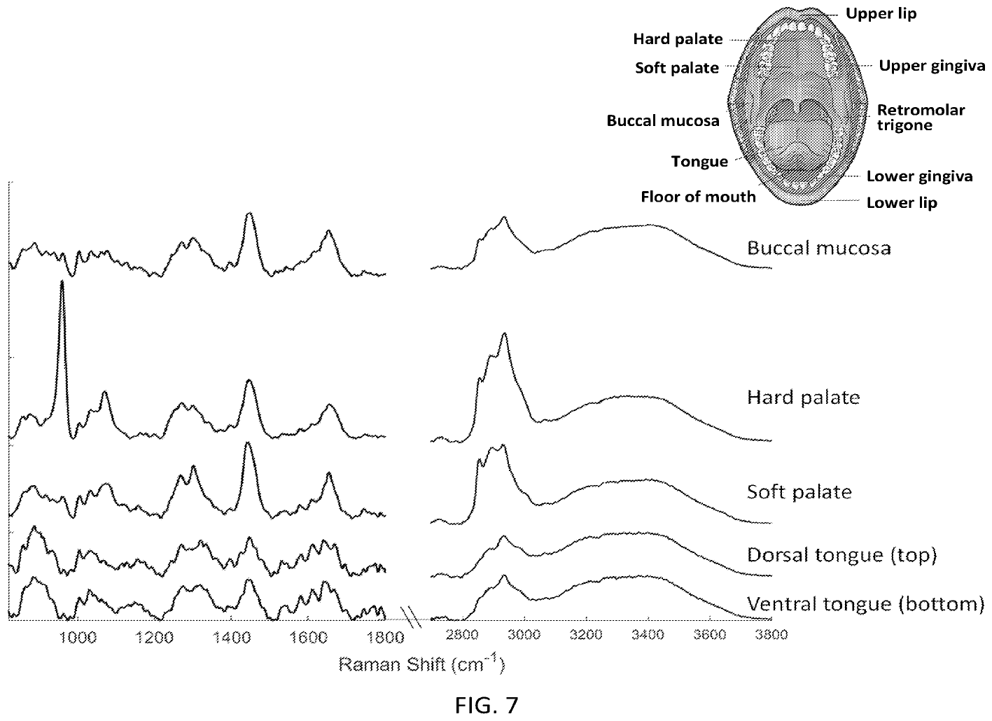

The described embodiment of the probe and the system has been tested on a subset of these potential applications. The first was to analyze the spectral signature of various oral tissues. The FP and HW spectra from a human subject are shown in FIG. 7.

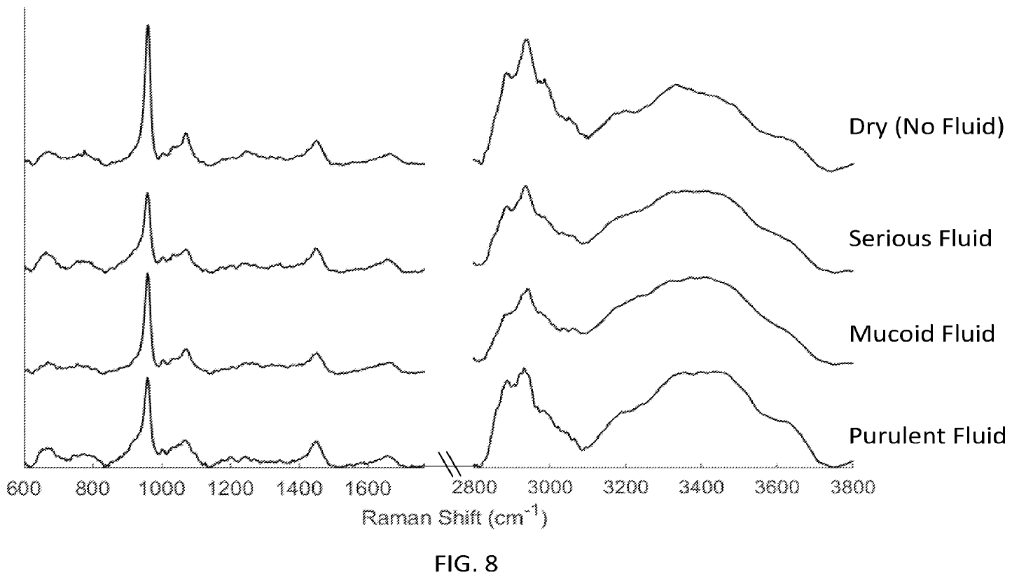

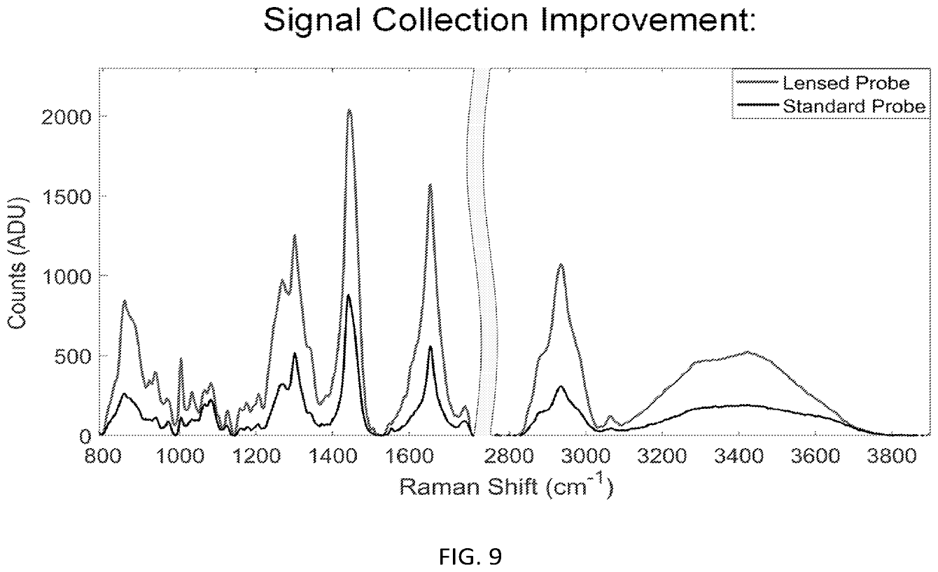

The second example was to analyze the Raman spectra of middle ear tissues in human subjects presenting with ear infections. The FP and HW spectra from two example cases, one with ear fluid and one without ear fluid, is shown in FIG. 8.

Briefly, the invention discloses, among other things, a novel small-diameter fiber optic probe using a single lens that does not interfere with the detected Raman signature from the sample while maintaining a small outer diameter. The fiber optic probe is also guided during device positioning to provide the user with feedback on orientation in the lateral and axial directions to repeatably measure specific locations on the sample. Probe guidance is given by either a miniature camera, optical interferometric imaging, or both.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

1. Fitzgerald, Sean, et al., “Multimodal Raman spectroscopy and optical coherence tomography for biomedical analysis.” Journal of Biophotonics 16.3 (2023): e202200231.
2. Monroy, Guillermo L., et al., “Multimodal handheld probe for characterizing otitis media—integrating Raman spectroscopy and optical coherence tomography.” Frontiers in Photonics 3 (2022): 929574.
3. Won, Jungeun, et al., “Pneumatic low-coherence interferometry otoscope to quantify tympanic membrane mobility and middle ear pressure.” Biomedical Optics Express, 9.2 (2018): 397-409.

1. A probe for collecting Raman signal of a target site of interest, comprising:
at least one first fiber operably coupled with a first light source and having a working end for delivering excitation light emitted from the first light source to the target site;
at least one second fiber operably coupled with a detector and having a working end for collecting Raman scattering light scattered from the target site in response to excitation by the excitation light to the detector; and
a lens positioned between the working ends of the at least one first fiber and the at least one second fiber and the target site for focusing the excitation light onto the target site and retrieving collection efficiency of the Raman scattering light.

2. The probe of claim 1, wherein the at least one second fiber includes a plurality of second fibers spatially arranged surrounding the at least one first fiber.

3. The probe of claim 2, wherein the at least one first fiber and the plurality of second fibers are spatially arranged in a row, a matrix, a wing, or a ring form.

4. The probe of claim 3, wherein the plurality of second fibers spatially is arranged in a radial ring form originated from the at least one first fiber.

5. The probe of claim 1, wherein the lens is adapted to minimize optical signal contribution from the lens itself that does not to interfere with Raman signature from the target site while maintaining a small outer diameter.

6. The probe of claim 5, wherein the lens is positioned at a distance of 2F from the working end of the at least second fiber, thereby providing an imaging relay with a working distance of 2F from the probe tip, wherein F is a focal length of the lens.

7. The probe of claim 5, wherein the lens includes a quartz lens, a sapphire lens, a calcium fluoride (CaF₂) lens, or a lens formed of any material whose inherent signal does not interfere with the Raman signals of the target site.

8. The probe of claim 1, further comprising a first optical filter placed at the working end of the at least first fiber and a second optical filter placed at the working end of the at least second fiber, respectively.

9. The probe of claim 8, wherein
the first optical filter is a short-pass or band-pass optical filter that blocks all wavelengths longer than that of the excitation light; and
the second optical filter is a long-pass or band-notch optical filter that blocks all wavelengths equal to or shorter than that of the excitation light, thereby preventing backscattered excitation light from being collected by the at least second fiber.

10. The probe of claim 9, wherein the first optical filter and the second optical filter are configured such that the probe is capable of measuring dual regions of the Raman scattering light from the target site, wherein the dual regions include a fingerprint (FP) region and a high-wavenumber (HW) region.

11. The probe of claim 10, wherein the dual regions of the Raman scattering light are sequentially acquired by switching the excitation light between a first wavelength and a second wavelength, wherein the first wavelength and the second wavelength are adapted such that when excited by the first wavelength light, the Raman scattering light corresponds to the FP region; and when excited by the second wavelength light, the Raman scattering light corresponds to the HW region.

12. The probe of claim 11, wherein the first wavelength is in a range of about 630-1064 nm, and the second wavelength is in a range of about 570-900 nm.

13. The probe of claim 12, wherein the first wavelength is about 785 nm, and the second wavelength about 680 nm, and wherein the short pass optical filter has a cut-off wavelength at about 785 nm, and the long pass filter has cut-on wavelength at about 800 nm.

14. The probe of claim 12, wherein the first wavelength is about 830 nm, and the second wavelength about 710 nm, and wherein the short pass optical filter has a cut-off wavelength at about 830 nm, and the long pass filter has cut-on wavelength at about 850 nm.

15. The probe of claim 9, wherein the first optical filter and the second optical filter are configured such that the probe is capable of measuring the Raman scattering light in a high-wavenumber (HW) region from the target site.

16. The probe of claim 15, wherein the lens includes a glass lens.

17. The probe of claim 1, further comprising a guidance mechanism for performing range sensing and providing feedback on orientation and position of the probe in lateral and axial directions to repeatably measure specific locations on the target site.

18. The probe of claim 17, wherein the guidance mechanism comprises a low-coherence interferometry (LCI) detector having at least one third fiber operably coupled with a second light source and having a working end for delivering low coherence light emitted from the second light source to the target site, wherein the at least one third fiber is located next to the at least one first fiber such that both beams of the excitation light and the low coherence light share the lens and are co-localized on the target site, ensuring that the LCI feedback is co-registered to the position of the excitation light spot.

19. The probe of claim 17, wherein the guidance mechanism comprises a miniature camera module located at the probe tip for providing wide-field visualization of the lateral position of the probe relative to the target site, wherein the miniature camera module includes a camera and an illumination fiber that delivers broadband light to visualize an imaging field of the camera.

20. The probe of claim 1, wherein the target site includes oral cavity and lymphoid tissues, middle ear tissues, cervical, gastrointestinal, esophageal and nasal tissues, or the like.

21. The probe of claim 1, being configured to measure the Raman scattering light without contact to the target site.

22. A system for assessment of a target site of interest, comprising:
a first light source configured to operably emit excitation light; and
a probe comprising:
at least one first fiber operably coupled with the first light source and having a working end for delivering the excitation light emitted from the first light source to the target site;
at least one second fiber having a working end for collecting Raman scattering light scattered from the target site in response to excitation by the excitation light; and
a lens positioned between the working ends of the at least one first fiber and the at least one second fiber and the target site for focusing the excitation light onto the target site and retrieving collection efficiency of the Raman scattering light.

23. The system of claim 22, further comprising a detector coupled with the probe for obtaining a plurality of Raman spectra from the collected Raman scattering light, wherein each Raman spectrum is associated with biomolecular content of a spot of the target site at which the Raman scattering light is scattered, and wherein the plurality of Raman spectra is processed to identify spectral features and assess the target site from the identified spectral features.

24. The system of claim 23, further comprising a controller operably coupled with the detector and configured to process the plurality of Raman spectra so as to identify spectral features and assess the target site from the identified spectral features.

25. The system of claim 24, further comprising a display operably coupled with the controller for displaying the plurality of Raman spectra, the identified spectral features, and/or the assessment of the target site.

26. The system of claim 22, wherein the first light source comprises a single wavelength laser module configured to operably emit the excitation light of a single wavelength, or a dual wavelength laser module configured to be operably emit the excitation light of a wavelength switchable between a first wavelength and a second wavelength.

27. The system of claim 22, wherein the at least one second fiber includes a plurality of second fibers spatially arranged surrounding the at least one first fiber.

28. The system of claim 24, wherein the at least one first fiber and the plurality of second fibers are spatially arranged in a row, a matrix, a wing, or a ring form.

29. The system of claim 28, wherein the plurality of second fibers spatially is arranged in a radial ring form originated from the at least one first fiber.

30. The system of claim 22, wherein the lens is adapted to minimize optical signal contribution from the lens itself that does not to interfere with Raman signature from the target site while maintaining a small outer diameter.

31. The system of claim 30, wherein the lens is positioned at a distance of 2F from the working end of the at least second fiber, thereby providing an imaging relay with a working distance of 2F from the probe tip, wherein F is a focal length of the lens.

32. The system of claim 30, wherein the lens includes a quartz lens, a sapphire lens, a calcium fluoride (CaF₂) lens, or a lens formed of any material whose inherent signal does not interfere with the Raman signals of the target site.

33. The system of claim 22, wherein the probe further comprises a first optical filter placed at the working end of the at least first fiber and a second optical filter placed at the working end of the at least second fiber, respectively.

34. The system of claim 33, wherein
the first optical filter is a short-pass or band-pass optical filter that blocks all wavelengths longer than that of the excitation light; and
the second optical filter is a long-pass or band-notch optical filter that blocks all wavelengths equal to or shorter than that of the excitation light, thereby preventing backscattered excitation light from being collected by the at least second fiber.

35. The system of claim 34, wherein the first optical filter and the second optical filter are configured such that the probe is capable of measuring dual regions of the Raman scattering light from the target site, wherein the dual regions include a fingerprint (FP) region and a high-wavenumber (HW) region.

36. The system of claim 35, wherein the dual regions of the Raman scattering light are sequentially acquired by switching the excitation light between the first wavelength and the second wavelength, wherein the first wavelength and the second wavelength are adapted such that when excited by the first wavelength light, the Raman scattering light corresponds to the FP region; and when excited by the second wavelength light, the Raman scattering light corresponds to the HW region.

37. The system of claim 36, wherein the first wavelength is in a range of about 750-1064 nm, and the second wavelength is in a range of about 630-750 nm.

38. The system of claim 37, wherein the first wavelength is about 785 nm, and the second wavelength about 680 nm, and wherein the short pass optical filter has a cut-off wavelength at about 785 nm, and the long pass filter has cut-on wavelength at about 800 nm.

39. The system of claim 37, wherein the first wavelength is about 830 nm, and the second wavelength about 710 nm, and wherein the short pass optical filter has a cut-off wavelength at about 830 nm, and the long pass filter has cut-on wavelength at about 850 nm.

40. The system of claim 35, wherein the first optical filter and the second optical filter are configured such that the probe is capable of measuring the Raman scattering light in a high-wavenumber (HW) region from the target site.

41. The system of claim 40, wherein the lens includes a glass lens.

42. The system of claim 22, wherein the probe further comprises a guidance mechanism for performing range sensing and providing feedback on orientation and position of the probe in lateral and axial directions to repeatably measure specific locations on the target site.

43. The system of claim 42, wherein the guidance mechanism comprises a low-coherence interferometry (LCI) detector having at least one third fiber operably coupled with a second light source and having a working end for delivering low coherence light emitted from the second light source to the target site, wherein the at least one third fiber is located next to the at least one first fiber such that both beams of the excitation light and the low coherence light share the lens and are co-localized on the target site, ensuring that the LCI feedback is co-registered to the position of the excitation light spot.

44. The system of claim 42, wherein the guidance mechanism comprises a miniature camera module located at the probe tip for providing wide-field visualization of the lateral position of the probe relative to the target site, wherein the miniature camera module includes a camera and an illumination fiber that delivers broadband light to visualize an imaging field of the camera.

45. The system of claim 22, wherein the probe is configured to measure the Raman scattering light without contact to the target site.

46. The system of claim 22, wherein the target site includes oral cavity and lymphoid tissues, middle ear tissues, cervical, gastrointestinal, esophageal and nasal tissues, or the like.

47. A non-transitory tangible computer-readable medium storing instructions which, when executed by one or more processors, cause the system of claim 22 to
deliver excitation light emitted from the first light source to the target site;
collect Raman scattering light scattered from the target site in response to excitation by the excitation light;
obtain a plurality of Raman spectra from the collected Raman scattering light; and
process the plurality of Raman spectra so as to identify spectral features and assess the target site from the identified spectral features.

48. The non-transitory tangible computer-readable medium of claim 47, wherein the instructions, when executed by the one or more processors, further cause the system to display the plurality of Raman spectra, the identified spectral features, and/or the assessment of the target site.
